(19) United States
(12) Patent Application Publication
(10) Pub. No.: US 2026/0174499 A1
(43) Pub. Date: Jun. 25, 2026

(54) AUTO PLANNING OF PROPOSED ABLATION TREATMENT

(71) Applicant: Neuwave Medical, Inc., Madison, WI (US)

(72) Inventor: Matthew SCHANING, Cambridge, WI (US)

(73) Assignee: Neuwave Medical, Inc., Madison, WI (US)

(21) Appl. No.: 18/947,362

(22) Filed: Nov. 14, 2024

(51) Int. Cl. A61B 18/18; A61B 18/00; A61B 34/00; A61B 34/10

(52) U.S. Cl. CPC A61B 18/1815; A61B 34/10; A61B 34/25; A61B 2018/00577; A61B 2018/1869; A61B 2034/104

(57) ABSTRACT

A system is disclosed that comprises an ablation probe that includes an antenna and a controller operable to receive an input indicative of a desired region to ablate with the ablation probe and automatically determine a proposed position of the ablation probe within the patient based on the input.

### BACKGROUND

Ablation procedures often exceed one hour in length, with most of the time focused on planning and placing ablation probes in desired positions, rather than the treatment itself. The planning and placement steps are crucial to ensure that healthy tissue beyond the desired margin of the intended target is not damaged. Due to the complexity of the planning and placement steps, ablation procedures typically have a high barrier to entry for interventional radiologists and other physicians.

Accordingly, systems and methods are desired that simplify, and decrease the time associated with, the planning and placement steps, while also minimizing collateral tissue damage to healthy tissue while still achieving desired margins.

### BRIEF DESCRIPTION OF THE DRAWINGS

The following figures are included to illustrate certain aspects of the present disclosure, and should not be viewed as exclusive embodiments. The subject matter disclosed is capable of considerable modifications, alterations, combinations, and equivalents in form and function, without departing from the scope of this disclosure.

FIG. 1 is a block diagram of an energy delivery system including an energy delivery device, in accordance with at least one aspect of the present disclosure.

FIG. 2 is a schematic diagram of the energy delivery device of FIG. 1, in accordance with at least one aspect of the present disclosure.

FIG. 3 is an enlarged, cross-sectional view of a portion of the energy delivery device of FIG. 2, in accordance with at least one aspect of the present disclosure.

FIGS. 4A and 4B are a flow diagram for automatically planning an ablation treatment for a patient, in accordance with at least one aspect of the present disclosure.

FIG. 5A is a graphical depiction of a first state of a display in which the display shows a two-dimensional pretreatment image that is overlaid with a two-dimensional image representative of a desired region to ablate, in accordance with at least one aspect of the present disclosure.

FIG. 5B is a second state of the display of FIG. 5A in which the display shows a three-dimensional pretreatment image that is overlaid with a three-dimensional image representative of the desired region to ablate, in accordance with at least one aspect of the present disclosure.

FIG. 6 is the display of FIG. 5A with the two-dimensional pretreatment image further overlaid with a proposed ablation zone, in accordance with at least one aspect of the present disclosure.

FIG. 7 is the display of FIG. 6 with the two-dimensional pretreatment image further overlaid with a proposed position of an ablation probe, in accordance with at least one aspect of the present disclosure.

FIG. 8 is the display of FIG. 5A with the two-dimensional pretreatment image further overlaid with an adjusted proposed ablation zone, in accordance with at least one aspect of the present disclosure.

FIG. 9A is the display of FIG. 8 with the two-dimensional pretreatment image further overlaid with an adjusted proposed position of an ablation probe, in accordance with at least one aspect of the present disclosure.

FIG. 9B is the second state of the display of FIG. 9A in which the display displays the three-dimensional pretreatment image that is overlaid with three-dimensional images representative of the desired region to ablate, the adjusted proposed ablation zone, and the adjusted proposed position of the ablation probe, in accordance with at least one aspect of the present disclosure.

### DETAILED DESCRIPTION

The present disclosure is related to systems and methods for delivering energy to tissue for ablation operation and, more particularly, to systems and methods for graphically visualizing an expected ablation treatment area mid-procedure.

The present disclosure is related to comprehensive systems, devices, and methods for delivering energy (e.g., microwave energy, radiofrequency energy, laser, focused ultrasound, plasma, etc.) to tissue for a wide variety of applications including medical procedures (e.g., percutaneous or surgical). Example medical procedures that may benefit from the embodiments described herein include, but are not limited to, tissue ablation, resection, cautery, vascular thrombosis, intraluminal ablation of a hollow viscus, cardiac ablation for treatment of arrhythmias, electrosurgery, tissue harvest, cosmetic surgery, intraocular use, or any combination thereof.

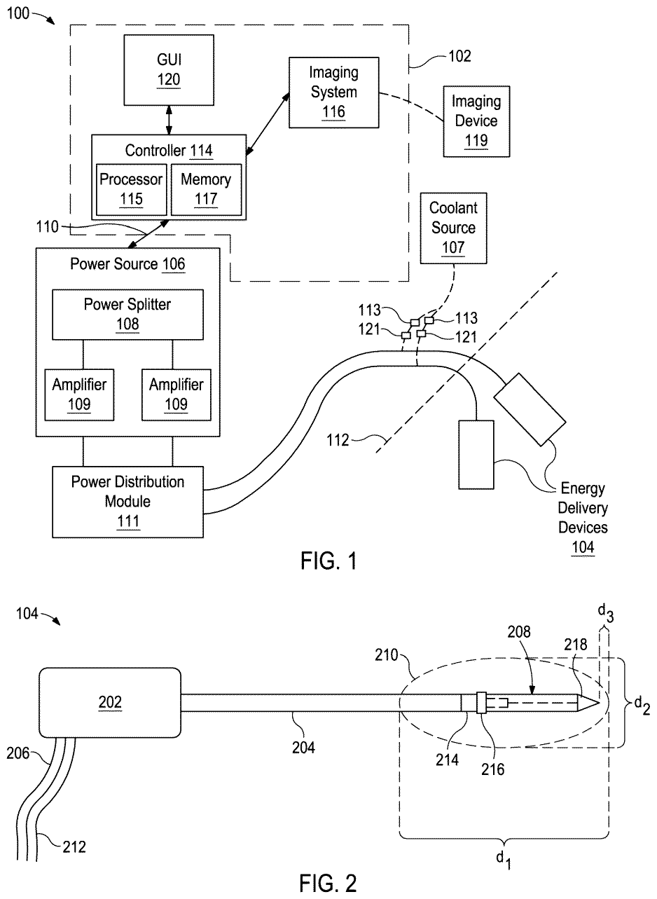

FIG. 1 is a block diagram of an energy delivery system 100, in accordance with at least one aspect of the present disclosure. As illustrated, the energy delivery system 100 (hereafter “the system 100”) may include a control system 102 and one or more energy delivery devices or “ablation probes” 104 (two shown) designed to deliver (emit) energy to a target tissue region of a patient. While two ablation probes 104 are shown, the system 100 may include only one ablation probe 104 or more than two ablation probes 104 (e.g. three, four, or five ablation probes).

The system 100 may further include a power source or generator 106 communicably coupled to the control system 102 and the ablation probes 104 to direct, control, and deliver (provide) electrical power thereto. The power source 106 may include a power splitter 108 that receives power from an external power source (e.g. a wall outlet) and directs power to one or more amplifiers 109 (two shown), which may amplify the voltage, current, or power from the power splitter 108 to an associated ablation probe 104. While two amplifiers 109 are shown, each associated with a corresponding ablation probe 104, the power source 106 may include less than two amplifiers (e.g. one amplifier) or more than two amplifiers (e.g. three, four, or five amplifiers, for example). Each amplifier 109 may be coupled to a corresponding ablation probe 104 via a power distribution module 111, which may provide strain relief to cabling extending from the amplifiers 109 to the ablation probes 104. The power distribution module 111 may be coupled to a structure in the operating room, such as a surgical bed, and may house connection hardware of the probes 104.

The power source 106 may supply energy required to operate various components of the system 100. The power source 106 may also supply energy to the ablation probes 104, such as microwave energy, radiofrequency energy, radiation, cryo energy, electroporation, high intensity focused ultrasound, or any combination thereof. In accordance with principles of the present disclosure, the power source 106 may supply microwave energy to the ablation probes 104 for purposes of tissue ablation. More specifically, power may be supplied to the ablation probes 104, but the microwave energy may be generated in a microwave generator and sent to the ablation probe 104. The power source 106 may include one or more energy generators configured to provide as much as 140-150 watts of microwave power at a frequency from 915 MHz to 5.8 GHz, although the present disclosure is not so limited. The power splitter 108 may comprise a power distribution system operable to distribute the energy from the power source 106 to the ablation probes 104. The power splitter 108 may be configured to provide varying energy levels to different regions of the ablation probes 104.

The control system 102 may monitor, control, and provide feedback concerning operation of the system 100. As illustrated, the control system 102 may include a controller 114, an imaging system 116, and a graphical user interface (GUI) or display 120, such as a touchscreen interface, which can be accessed by a user (e.g., a surgeon, a nurse, bedside assist, etc.) to operate the system 100. In some applications, the control system 102 may be mounted to or otherwise form part of a portable cart or “procedure cart,” and the GUI 120 may be arranged in a display region for operating and/or monitoring the components of the system 100.

The controller 114 may include a processor 115 and a memory or memory device 117 comprising any storage media readable by the processor 115. The memory 117 may store software or software instructions executable by the processor 115 to carry out functions and operations of the system 100. Examples of the memory 117 include, but are not limited to, random access memory (RAM), read-only memory (ROM), computer chips, optical discs (e.g., compact discs (CDs), digital video discs (DVDs), etc.), magnetic disks (e.g., hard disk drives (HDDs), floppy disks, ZIP® disks, etc.), magnetic tape, and solid state storage devices (e.g., memory cards, “flash” media, etc.). As used herein, the term “computer readable medium” refers to any device or system for storing and providing information (e.g., data and instructions) to the processor 115. Examples of computer readable media include, but are not limited to, optical discs, magnetic disks, magnetic tape, solid-state media, and servers for streaming media over networks.

Based on instructions provided by the software, the controller 114 may be configured to regulate the amount of energy (e.g., microwave energy) provided to a tissue region by the ablation probes 104 by monitoring characteristics of the tissue region, such as the size and shape of a target tissue, the temperature of the tissue region, etc. The controller 114 interacts with the ablation probes 104 to raise or lower (e.g., tune) the amount of energy delivered to the tissue region. The controller 114 may also be configured to prime coolants for distribution into the ablation probes 104 such that the coolant is delivered at a desired temperature, as discussed in more detail below.

In some applications, the type of tissue being treated is inputted into the software for purposes of allowing the controller 114 to regulate (e.g., tune) the delivery of microwave energy to the tissue region based upon pre-calibrated methods for that particular type of tissue or tissue region. In other embodiments, however, the type of probe selected for the particular procedure may be specifically tuned to a specific tissue type, and projected (expected) ablation sizes may be based on tissue type. In such embodiments, the controller 114 may not control power delivery based on tissue type. In yet other embodiments, the controller 114 generates a chart or diagram based upon a particular type of tissue or tissue region displaying characteristics useful to a user of the system.

The controller 114 may allow a user to choose power, duration of treatment, different treatment algorithms for different tissue types, simultaneous application of power to multiple probes 104, coherent and incoherent phasing, etc. The controller 114 may also be configured to create a database of information (e.g., required energy levels, duration of treatment for a tissue region based on particular patient characteristics, etc.) pertaining to ablation treatments for a particular tissue region based upon previous treatments with similar or dissimilar patient characteristics.

The imaging system 116 may be in communication with the controller 114 and comprise one or more imaging devices 119. Example imaging devices include, but are not limited to, ultrasound transducers, endoscopic devices, stereotactic computer assisted neurosurgical navigation devices, thermal sensor positioning systems, motion rate sensors, steering wire systems, intraprocedural ultrasound, interstitial ultrasound, microwave imaging, acoustic tomography, dual energy imaging, fluoroscopy, computerized tomography magnetic resonance imaging, nuclear medicine imaging devices triangulation imaging, thermoacoustic imaging, infrared and/or laser imaging, or electromagnetic imaging. In some embodiments, the system 100 uses endoscopic cameras, imaging components, and/or navigation systems that permit or assist in placement, positioning, and/or monitoring of the ablation probes 104.

The imaging system 116 may be configured to monitor ablation procedures, such as a position of the ablation probes 104 within a patient, as described in more detail below, and/or the amount of ablation occurring within a particular tissue region(s) undergoing a thermal ablation procedure. The monitoring includes, but is not limited to, MRI imaging, CT imaging, ultrasound imaging, nuclear medicine imaging, and fluoroscopy imaging. The software may be designed to automatically obtain images of a tissue region (e.g., MRI imaging, CT imaging, ultrasound imaging, nuclear medicine imaging, fluoroscopy imaging), automatically detect any changes in the tissue region (e.g., blood perfusion, temperature, amount of necrotic tissue, etc.), and based on the detection to automatically adjust the amount of energy delivered to the tissue region through the ablation probes 104.

The components of the system 100 may be connected via one or more cables or transmission lines 110. Moreover, the ablation probes 104 are designed to operate within a sterile field facilitated by the use of a sterile field barrier 112 that separates the ablation probes 104 from the remaining components of the system 100. The sterile field barrier 112 creates the sterile field, which includes any region permitting access only to sterilized items (e.g., sterilized devices, sterilized accessory agents, sterilized body parts, etc.). The sterile field barrier 112 hinders entry of non-sterile items into the sterile field, and the ablation probes 104 are configured for operation within the sterile field.

The system 100 may further include a coolant source 107, which stores therein a cooling fluid or “coolant”. Example coolants include, but are not limited to, water, glycol, air, inert gases (e.g., helium), carbon dioxide, nitrogen, sulfur hexafluoride, ionic solutions (e.g., sodium chloride with or without potassium and other ions), dextrose in water, Ringer's lactate, organic chemical solutions (e.g., ethylene glycol, diethylene glycol, or propylene glycol), oils (e.g., mineral oils, silicone oils, fluorocarbon oils), liquid metals, freons, halomethanes, liquified propane, other haloalkanes, anhydrous ammonia, sulfur dioxide, or any combination thereof.

The system 100 may further include one or more valves 113 fluidically coupled to the coolant source 107 and a respective ablation probe 104. The valves 113 may control the flow rate and/or pressure of coolant from the coolant source 107 to the respective ablation devices 104. The valves 113 may be any suitable valve (e.g., gate, globe, ball, etc.) that is transitionable (actuatable) between an open state (e.g., 100% open), a closed state (e.g., 0% open), and a plurality of partially open states between the open and closed states (e.g., 10%, 25%, 50%, 75%, or 90%). In applications where the valves 113 are electromechanically actuatable, the valves 113 may each include a motor in operable communication with the controller 114, via a wired or wireless connection, and which function to transition the valves 113 between their respective open, closed, and partially open states. Alternatively, the valves 113 may be solenoid valves that are transitionable between their respective open, closed, and partially opened states by the controller 114. The valves 113 may be transitionable between their respective open, closed, and partially open states based on a user input provided to the GUI 120 or automatically, such as based on inputs provided to the controller 114 from various sensors of the system 100.

The system 100 may further include one or more sensors 121 for sensing one or more parameters associated with the coolant provided from the coolant source 107. The sensors 121 may include pressure sensors operable to sense a pressure of the coolant provided from the coolant source 107, flow sensors operable to sense a flow rate of the coolant provided from the coolant source 107, or temperature sensors for sensing a temperature of the coolant provided from the coolant source 107, or any combination thereof. Each valve 113 may have associated therewith one or more of the sensors 121 to sense one or more parameters of the coolant provided to the respective ablation probe 104. The controller 114 may be in operable communication with the one or more sensors 121, via a wired or wireless connection, and may receive the sensed parameters therefrom. The controller 114 may control one or more aspects of the system 100 based on the sensed parameters, such as the state of the valves 113.

FIG. 2 is a schematic diagram of an example ablation probe 104, in accordance with at least one aspect of the present disclosure. As indicated above, the ablation probe 104 may be configured to deliver (emit) energy (e.g., microwave energy, radiofrequency energy, radiation energy) to a target tissue region. As illustrated, the ablation probe 104 includes a handle or housing 202 and an elongate shaft or probe cannula 204 extending distally from the handle 202.

A cable or cable assembly 206 may be operatively coupled to the handle 202 and configured to convey electrical power thereto. The cable assembly 206 may extend from the power distribution module 111 (FIG. 1), for example, and may provide the power sufficient to operate the ablation probe 104. An antenna 208 is provided at the distal end of the probe cannula 204 and receives electrical power from the cable assembly 206 to emit energy (e.g., microwave energy) to a target tissue region and thereby generate an ablation zone 210 (shown in dashed lines).

The ablation zone 210 may include a longitudinal length d₁, a lateral width d₂ (e.g. a diameter of the ablation zone 210), and a distance d₃ between a distal end of the stylet 218 and a distal-most end of the ablation zone 210. The size of the dimensions d₁, d₂, d₃ of the ablation zone 210 may depend on one or more parameters, such as the type of ablation probe, the power level P of the power source 106, and the amount of time T the ablation probe 104 is energized. For instance, a first type of ablation probe 104 that is energized at a first power level P₁ for a first amount of time T₁ may be expected to generate an ablation zone 210 that includes a first longitudinal length (d₁), a first lateral width (d₂) and a first distance (d₃) between the distal end of the stylet 218 and the distal-most end of the ablation zone, while a second ablation probe 104 different than the first ablation probe that is energized at a second power level P₂ different than the first power level P₁ for a second amount of time T₂ different than the first amount of time T₁ may be expected to generate an ablation zone that includes a second longitudinal length (d₁′) different from the first longitudinal length (d₁), a second lateral width (d₂′) different from the first lateral width (d₂), and a second distance (d₃′) between the distal end of the stylet 218 and the distal-most end of the ablation zone different from the first distance (d₃). It should be noted that only one of the parameters described above (e.g. type of instrument, power level, and time) may need to be adjusted to change the resulting dimensions d₁, d₂, d₃.

The memory 117 may store therein a look-up table that includes various combinations of types of ablation probes, activation times T, and power levels P that are expected to yield various sized ablation zones 210 with varying dimensions (e.g. d₁, d₂, d₃). The expected dimensions of the ablation zone may be based on ex-vivo data, in-vivo data, or clinical data, or combinations thereof. Accordingly, should a user desire to generate an ablation zone 210 with particular dimensions, the controller 114 may retrieve, from the memory 117, a particular combination of type of ablation probe, activation time T, and power level P that is expected to yield an ablation zone that meets, or at least substantially comes close to, the dimensions desired of the user.

A cooling tube 212 may be operatively coupled to the coolant source 107 (FIG. 1), and the handle 202 and may be configured to convey the coolant from the coolant source 107 to the ablation probe 104.

The ablation probe 104 may include a sharp stylet tip or “stylet” 218 located at the distal end of the antenna 208 and otherwise forming the distal end of the ablation probe 104. The stylet 218 may facilitate percutaneous insertion of the ablation probe 104. The stylet 218 may be made of a variety of rigid or hardened materials including, but not limited to, a hardened resin, a metal (e.g., titanium or an equivalent of titanium, stainless steel, etc.), a ceramic, or any combination thereof. In at least one application, the stylet 218 may be brazed to zirconia or an equivalent of zirconia. In such applications, the stylet 218 may comprise an extension of a metal portion of the antenna 208 and may be electrically active.

The ablation probe 104 may further include a stick region 214, alternately referred to as a “tissue-loc” region, provided on the probe cannula 204 and a plug region 216 provided on the probe cannula 204 distal to the stick region 214 at or near the antenna 208. The stick and plug regions 214, 216 may be defined as portions of the probe cannula 204, and will be discussed in more detail below.

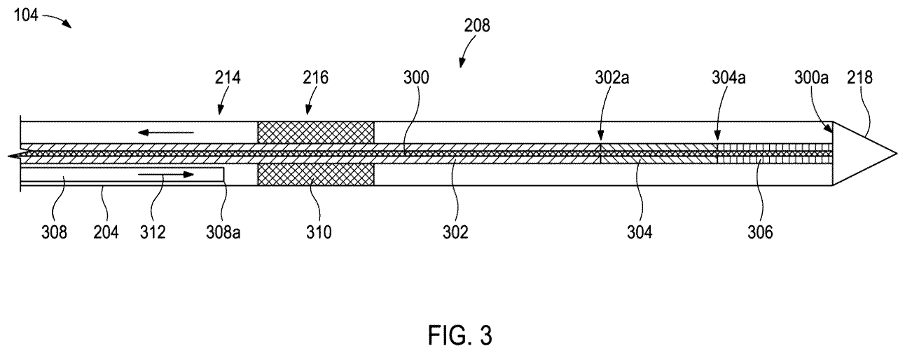

FIG. 3 is an enlarged, cross-sectional view of the probe cannula 204 and antenna 208 of the ablation probe 104, in accordance with at least one aspect of the present disclosure. The ablation probe 104 may include an inner conductor 300 and an outer conductor 302 extending through the probe cannula 204 and the antenna 208, with the outer conductor 302 being positioned about (around) the inner conductor 300 (i.e., the inner conductor 300 extends within the outer conductor 302). As illustrated, a distal end 300a of the inner conductor 300 extends distal to (beyond) a distal end 302a of the outer conductor 302. The inner and outer conductors 300, 302 may be made of a conductive material that allows current to be transmitted along their lengths thereof. The inner and outer conductors 300, 302 may be made of a metal, for example, such as stainless steel, silver, copper, brass or aluminum, or alloys thereof.

The ablation probe 104 may further include an insulator 304 extending from the distal end 302a of the outer conductor 302 and positioned about (around) the inner conductor 300. The insulator 304 may be made of a variety of non-conductive materials including, but not limited to, a ceramic or a polymer, such as polyamide, linear polyethylene (PE), polyethylene terephthalate (PET), polytetrafluoroethylene (PTFE) isotactic polypropylene (PP), or a polymer in the polyaryletherketone (PAEK) family, such as polyetheretherketone (PEEK), as examples.

The ablation probe 104 may further include a conductor load 306 extending from a distal end 304a of the insulator 304 and positioned about (around) the inner conductor 300. Accordingly, the insulator 304 axially interposes the outer conductor 302 and the conductor load 306. The conductor load 306 may be made of a metal, such as stainless steel, silver, copper, brass or aluminum, alloys thereof, or any combination thereof. The conductor load 306 may serve as a load point and may include a longitudinal length that is tuned to match the dielectric properties of the surrounding tissue.

The ablation probe 104 may further include a cooling tube 308 extending through the probe cannula 204 and terminating at the stick region 214. The cooling tube 308 may be fluidically coupled to the cooling tube 212 (FIG. 2) such that the cooling tube 308 may convey coolant 312 from the coolant source 107 (FIG. 1) and the cooling tube 212 (FIG. 2) to the stick region 214. A user may provide an input to the handle 202 (FIG. 2) and/or the GUI 120 (FIG. 1) to control flow of the coolant 312 through the cooling tube 308 to regulate a temperature of the antenna 208, the ablation zone 210, and/or the stick region 214, as discussed in more detail below. The cooling tube 308 may be made of a polymer, such as polyamide, linear polyethylene (PE), polyethylene terephthalate (PET), polytetrafluoroethylene (PTFE) isotactic polypropylene (PP), or a polymer in the polyaryletherketone (PAEK) family, such as polyetheretherketone (PEEK), as examples.

The stick region 214 is designed to attain and maintain a temperature that promotes adherence of a tissue region onto the surface of the stick region 214. More specifically, the stick region 214 may operate as an anchoring element that freezes the interface between the stick region 214 and the adjacent tissue, thereby sticking (maintaining, locking, etc.) the antenna 208 in place. In operation, coolant 312 from the coolant source 107 (FIG. 1) may be conveyed to the stick region 214 by way of the cooling tube 212 (FIG. 2) and expelled from a distal end 308a of the cooling tube 308. Upon expulsion from the distal end 308a of the cooling tube 308, the pressure of the coolant 312 quickly decreases, thereby causing the coolant 312 to correspondingly decrease in temperature (the Joule-Thompson effect). The decreased temperature of the coolant 312 causes the temperature of the stick region 214 to decrease, and the coolant 312 is recirculated back along the probe cannula 204, such as to a coolant sink, for example.

Once a threshold “low” temperature is reached at the stick region 214, contact with adjacent tissue causes the tissue to adhere (stick or couple) to the stick region 214, thereby resulting in attachment of the energy delivery device 104 to the tissue. During ablation, as the tissue warms, the antenna 208 remains secured to the tissue region due to tissue desiccation and charring. The stick region 214 may be made of any material able to attain and maintain a temperature such that contact with tissue results in adherence of the tissue onto the stick region 214. Example materials for the stick region 214 include, but are not limited to, a metal.

With reference now to FIGS. 1 and 3, the controller 114 may control a state of one or more of the valves 113, thereby controlling an amount of coolant 312 provided to the stick region 214 of the ablation probe 104. Tissue may not adhere to the stick region 214 unless a threshold flow rate and/or pressure of coolant 312 is conveyed through the cooling tube 308 to the stick region 214. For instance, the valves 113 may be transitionable between a first state and a second state. The first state may be a first, partially opened state of the valve 113, where the valve 113 is opened a first amount, and the second state may be a second, partially opened state or the open state of the valve 113, where the valve 113 is opened a second amount greater than the first amount. The first and second states of the valves 113 may be stored in the memory 117.

In the first state, a first flow rate and/or pressure of coolant 312 may be conveyed through the cooling tube 308 to the stick region 214. The first flow rate and/or pressure of coolant 312 may be insufficient to cause tissue to adhere to the stick region 214, thereby allowing the stick region 214 to move relative to the tissue positioned thereagainst. In the second state, a second flow rate and/or pressure of coolant 312 greater than the first flow rate and/or pressure of coolant 312 may be conveyed through the cooling tube 308 to the stick region 214. The second flow rate and/or pressure of coolant 312 may be sufficient to cause tissue to adhere to the stick region 214, thereby preventing the stick region 214 from moving relative to the tissue positioned thereagainst.

The ablation probe 104 may further include a seal 310, which may define the plug region 216 on the probe cannula 204. As illustrated, the seal 310 may be provided distal to the distal end 308a of the cooling tube 308 and the stick region 214 and otherwise interposing the stick region 214 and the antenna 208. The seal 310 may be configured to prevent a reduction in temperature resulting from the cooled probe cannula 204 and the stick region 214 from affecting (e.g., reducing) the temperature within the antenna 208. Accordingly, the seal 310 separates interior portions of the ablation probe 104 to prevent cooling or heating of a portion or portions of the probe 104 while permitting cooling or heating of other portions. The seal 310 may be made of an insulative material capable of being in contact with a material or region having a low temperature without having its temperature significantly reduced. Example insulative materials for the seal 310 include, but are not limited to, a synthetic polymer (e.g., polystyrene, polyicynene, polyurethane, polyisocyanurate), aerogel, fiberglass, cork, or any combination thereof.

Additional information regarding the ablation probe 104, such as the construction and function thereof, is described in U.S. Pat. No. 11,638,607, entitled “ENERGY DELIVERY SYSTEMS AND USES THEREOF”, which issued on May 2, 2023, the contents of which are hereby incorporated by reference in their entirety herein.

With continued reference to FIGS. 1 and 3, the controller 114 may be operable to use the coolant source 107 and the coolant stored therein to reduce undesired heating within and along the ablation probes 104. In particular, the controller 114 may control conveyance (flow) of the coolant into and out of the cooling tube 308 via the valves 113. The controller 114 may also be configured to control conveyance of coolant to the stick region 214, as described herein above to thereby attain and maintain a temperature that accommodates adherence of tissue onto the surface of the stick region 214. In some embodiments, a user may provide an input to the controller 114, such as via the GUI 120. Based on the input, the controller 114 may control a state of one or more of the valves 113, as discussed herein above, thereby allowing the coolant source 107 to provide coolant to the stick region 214 via the cooling tubes 212, 308.

The controller 114 may also be operable to continuously or intermittently monitor the real-time temperature of the ablation probes 104. In such embodiments, the controller 114 may communicate with one or more temperature sensors (e.g., thermocouples) terminating at various points along the probe cannula 204 and/or the antenna 208 (FIG. 2) of the ablation probe 104. Consequently, localized temperature may be monitored at several points along the antenna 208 to estimate ablation status, cooling status, or safety checks. In some applications, monitoring the temperature at several points along the antenna 208 may help determine the geographical characteristics of the ablation zone 210, such as diameter, depth, length, density, width, etc., based upon the tissue type, and the amount of power used in the ablation probe 104. In other embodiments, or in addition thereto, the temperature may be measured not only at specific points along the probe cannula 204, but continuously along its entire length.

In some embodiments, the probe cannula 204 includes a plurality of temperature sensors. A first temperature sensor may be placed at, or slightly proximal to, the antenna 208 to provide real-temperature measurements of the tissue being heated by the antenna 208. A second temperature sensor may be placed at, or adjacent to, the stick region 214 to provide real-time temperature measurements of the tissue that is being cooled, and thus adhered to, the stick region 214. A third temperature sensor may be located proximal to the first and second temperature sensors along the cannula 204, such as at the point of entry into the skin, to provide real-time measurements of the patent's skin. The control system 102 can receive the temperature measurements from the first, second, and third sensors to control the coolant systems and cooling fluids from the controller 114 to the stick region 214 and/or other cooling systems of the energy delivery device 104.

The controller 114 may also be operable to monitor the temperature of a tissue region (e.g., tissue being treated, surrounding tissue). This may prove advantageous in helping to determine the status of the procedure (e.g., the end of the procedure). The controller 114 may communicate with the plurality of temperature sensors to provide real-time temperature information to a user and display such measurements on the GUI 120. In at least one embodiment, based on the temperature data obtained by the controller 114, the controller 114 may be configured to autonomously adjust operation of the system 100 appropriately.

### Planning of Ablation Treatment Zone and Probe Insertion Path

Ablation procedures often exceed one hour in length, with most of the time focused on planning and placing ablation probes (e.g., ablation probes 104) in desired positions, rather than the treatment itself. The planning and placement steps are crucial to ensure that healthy tissue beyond the desired margin of the intended target is not damaged. Due to the complexity of the planning and placement steps, ablation procedures typically have a high barrier to entry for interventional radiologists and other physicians. Accordingly, systems and methods are desired that simplify and decrease the time associated with the planning and placement steps, while also minimizing collateral tissue damage to healthy tissue while still achieving desired margins.

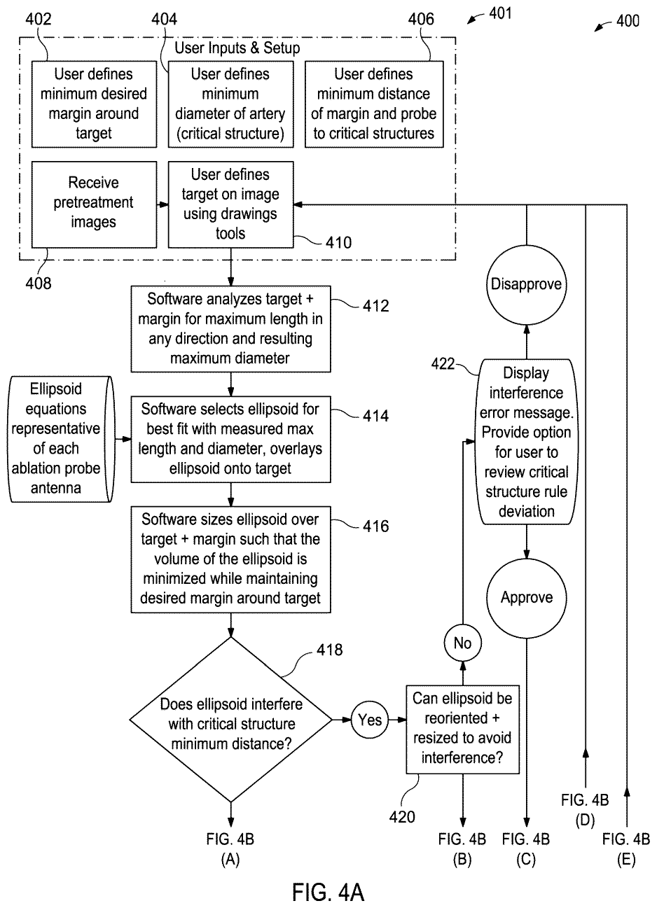

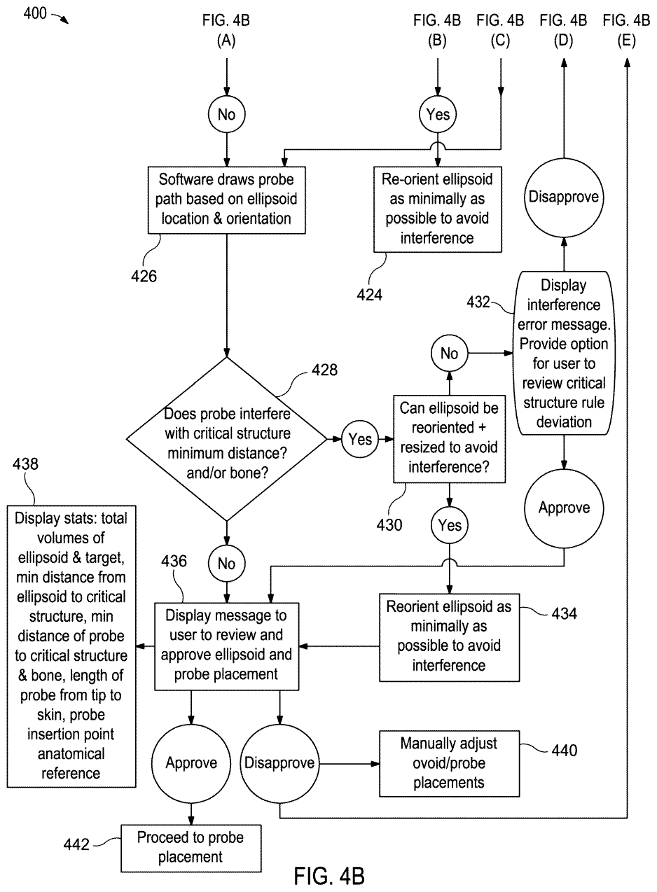

FIGS. 4A and 4B provide a schematic flow diagram 400 for automatically planning an ablation treatment or procedure for a patient, according to at least one aspect of the present disclosure. The flow diagram 400 may be embodied as an algorithm, stored in the memory 117 (FIG. 1) of the controller 114 (FIG. 1), and may be executed by the processor 115 (FIG. 1), such as based on an input provided to the controller 114 (FIG. 1) by a user.

With reference to FIGS. 1 and 4A, the controller 114 may receive one or more “set-up” parameters, as at step 401. The controller 114 may receive the set-up parameters based on a user providing one or more inputs to the controller 114, such as via the GUI 120. Alternatively, the set-up parameters may be predefined and stored in the memory 117, and may be retrievable by the controller 114. The predefined set-up parameters stored in the memory 117 may be adjusted by the user based on an input provided to the controller 114, such as via the GUI 120. The set-up parameters may include a desired margin 402, a desired minimum artery diameter to be identified as a critical structure 404, a desired minimum distance from critical structures 406, or combinations thereof. The set-up parameters may be stored, permanently or temporarily, in the memory 117, and may be retrievable by the controller 114 during the algorithm 400, as will be discussed in more detail below.

The controller 114 may further receive one or more pretreatment images of a patient, as at step 408. The pretreatment images may be MRI images, CT images, ultrasound images, nuclear medicine images, fluoroscopy images, or combinations thereof, which may be captured by the imaging device 119. The pre-treatment images may be two-dimensional (2D) or a three-dimensional (3D) images, or combinations thereof. The pretreatment images may be provided to the controller 114, such as from the imaging device 119 via the imaging system 116. The controller 114 may display one or more of the pretreatment images on the GUI 120 for a user to view.

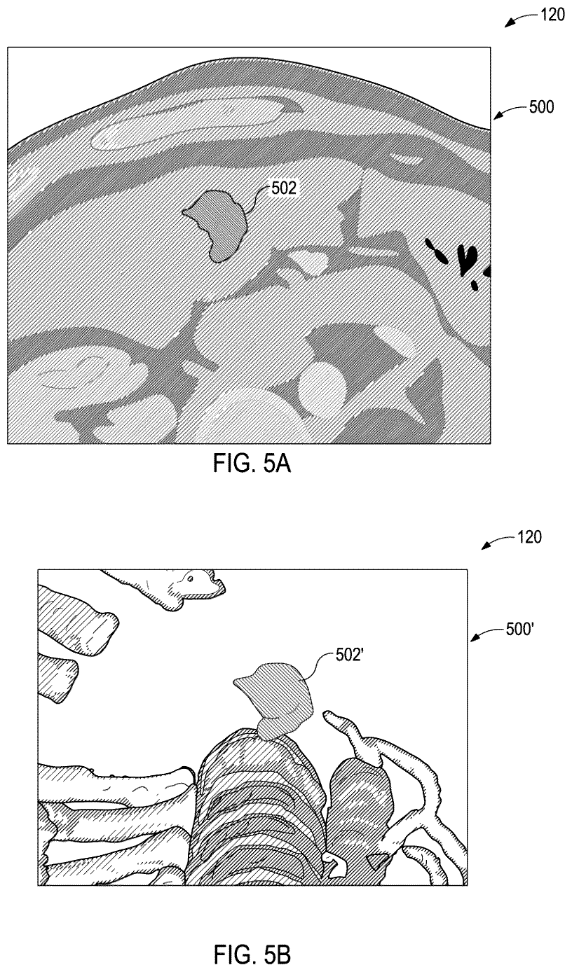

The controller 114 may further receive a user input indicative of a desired region to ablate, as at step 410. For instance, referring to FIG. 5A, the controller 114 may display, on the GUI 120, a pretreatment image 500 obtained at step 408, such as a two-dimension (2D) CT image. The user may digitally draw on the pretreatment image 500, such as with a mouse or stylet, for example, a desired region to ablate. Based on receiving the input indicative of the desired region to ablate, the controller 114 may overlay, on the displayed pretreatment image 500, an image 502 representative of the desired region to ablate.

In some applications, the user may desire to view the image 502 indicative of the desired region to ablate from an alternative view to determine if the desired region to ablate is satisfactory. To accomplish this, a user may provide an input to the controller 114 to transition the GUI 120 between a first state, in which the controller 114 displays, on the GUI 120, a first pre-treatment image (e.g. the 2D CT image 500 with the overlaid 2D image 502 (FIG. 5A)), and a second state, in which the controller 114 displays, on the GUI 120, a second pre-treatment image different than the first pre-treatment image (e.g. a 3D CT image 502′ with a 3D image 502′ indicative of the desired region to ablate overlaid on the 3D CT image 500′, as shown in FIG. 5B). Accordingly, a user may transition the GUI 120 between the first and second states to determine if the desired region to ablate is satisfactory.

Should a user find the desired region to ablate unsatisfactory, the user may provide an input to the controller 114 to remove the images 502, 502′ from pretreatment images 500, 500′, thereby allowing the user to redraw another desired region to ablate (step 410).

Based on overlaying the image 502, the controller 114 may further overlay, on the pretreatment image 500, an image representative of the desired margin around the desired region to ablate. For instance, the controller 114 may retrieve, from the memory 117, the desired margin provided to the controller 114 at step 402 and overlay, on the pretreatment image, the desired margin about the desired region to ablate. The desired margin may be the area/volume surrounding the desired region to ablate to ensure that all (or substantially all) of the desired region to ablate is ablated. The desired region to ablate and the desired margin thereabout will hereinafter be referred to collectively as the “desired ablation target” with the desired margin defining a perimeter (boundary) of the desired ablation target.

The controller 114 may further determine the maximum length and diameter of the desired ablation target, as at step 412. The memory 117 may store therein software that measures distances from pretreatment images, and the controller 114 may utilize the stored software to determine the maximum length and diameter of the desired ablation target. Based on the dimensions determined at step 412, the controller 114 may proceed with selecting a proposed ablation zone, determining an orientation thereof that is expected to encompass (or at least substantially encompass) the desired ablation target, and overlaying an image of the proposed ablation zone on the pretreatment image in the proposed orientation, as at steps 414 and 416.

For instance, as previously discussed, the memory 117 may store therein various combinations of different types of ablation probes, activation times T, and power levels P that are expected to yield various different sized ablation zones with various dimensions (e.g. d₁, d₂, d₃; see FIG. 2). With respect to the ablation probes, the different types of ablation probes may be energizable (i.e., able to be energized or powered) to generate various shaped ablation zones. For instance, a first ablation probe may be energizable to generate a spherical ablation zone in which d₁ is substantially similar to d₂, while a second ablation probe may be energizable to generate an elongated ablation zone, similar to what is shown in FIG. 2, in which d₁ is greater than d₂. The controller 114 may select the ablation probe and corresponding shape at step 414. Based on selecting the ablation shape, the controller 114 may retrieve, from the memory 117, a combination of the type of ablation probe, a power level P, and a time T that is expected to yield an ablation zone that may be oriented (sized) to encompass (or at least substantially encompass) the desired ablation target at step 416. The longitudinal length d₁ and the lateral width d₂ (e.g. the diameter of the proposed ablation zone 210) of the proposed ablation zone may be selected based on the maximum distance and diameter of the desired ablation zone, as determined at step 412. The controller 114 may size the proposed ablation zone such that the volume of the proposed ablation zone is minimized while maintaining the desired margin around the desired ablation target. In some embodiments, steps 414 and 416 are completed as one step as opposed to two separate steps.

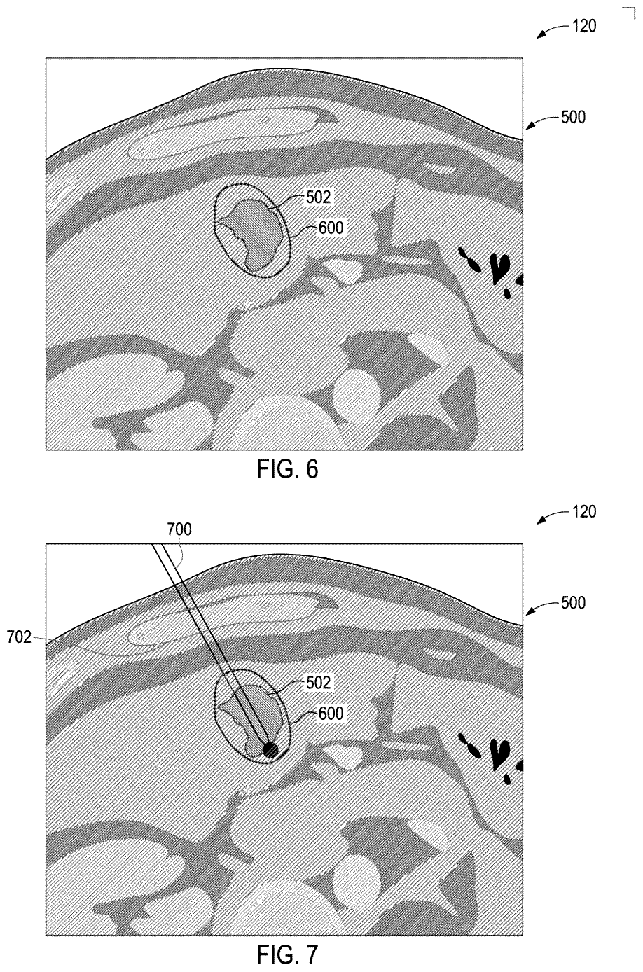

Referring now to FIG. 6, with continued reference to FIGS. 1 and 4A, based on selecting the proposed ablation zone at steps 414 and 416, the controller 114 may overlay, on the pretreatment image 500 around the image 502 of the desired region to ablate, an image 600 representative of the proposed ablation zone.

The controller 114 may then proceed with determining if the proposed ablation zone traverses (interferes with) or is otherwise within a predetermined threshold distance of a critical structure, as at step 418. The critical structures may be identified based on the desired minimum artery diameter provided to the controller 114 at step 404 The controller 114 may determine if any critical structures (e.g., veins, arteries, nerves, ducts, or other surgical instruments) are present within the pretreatment images. The controller 114 may identify these critical structures using the imaging device 119 or with image recognition software stored in the memory 117. Based on determining the presence of critical structures in the patient, the controller 114 may determine distances between the perimeter (boundary) of the proposed ablation zone and each of the critical structures. The controller 114 may retrieve, from the memory 117, the desired minimum distance from critical structures, provided to the controller 114 at step 406, and compare the desired minimum distance to the measured distances.

Based on determining that no critical structures are within the desired minimum distance, the controller may proceed to step 426, discussed in more detail below with reference to FIG. 4B. Based on at least one of the measured distances being at or less than the desired minimum distance, the controller 114 may proceed with determining if the proposed ablation zone can be adjusted (e.g. re-oriented and/or re-sized with a different combination of parameters (type of probe, power P, or time T)) such that the proposed ablation zone still encompasses, or at least substantially encompasses, the desired ablation target and has a boundary that is a distance greater than the desired minimum distance away from the identified critical structures, as at step 420.

It should be noted that adjusting the ablation zone may result in a larger proposed ablation zone that may cause additional collateral damage to healthy, non-targeted, tissue. This adjustment, however, may preserve critical structures while still encompassing the desired ablation target. Accordingly, the adjustment to the proposed ablation zone may be preferable despite the additional collateral damage to healthy, non-targeted tissue that may be expected.

Based on the controller 114 determining that the proposed ablation zone can be adjusted at step 420, the controller 114 may proceed with adjusting the proposed ablation zone as minimally as possible, as at step 424. If the controller 114 determines that the proposed ablation zone cannot be adjusted, the controller 114 may proceed with providing an alert (e.g., a visual alert via the GUI 120) indicating to a user that an ablation zone cannot be proposed with the provided inputs.

The controller 114 may determine an adjustment to one or more of the provided set-up parameters (e.g. the margin or the minimum distance) that would allow the controller 114 to continue with the algorithm 400. For instance, the controller 114 may propose a first adjustment (decrease) to one or more set-up parameters that would allow the controller 114 to proceed directly to step 426 (FIG. 4B). The controller 114 may also propose a second adjustment (decrease) to one or more set-up parameters different (less) than the first adjustment, thereby allowing the controller 114 to adjust the proposed ablation zone at step 424 (FIG. 4B).

A user may provide an input to the controller 114, via the GUI 120, in response to the proposals displayed on the GUI 120 at step 422. For instance, a user may provide an input to the controller 114 indicating that the proposed adjustments are disapproved. Accordingly, the controller 114 may proceed back to step 410 and indicate to the user, via the GUI 120, that a new desired ablation zone must be drawn to satisfy the desired set-up parameters. Alternatively, a user may provide an input to the controller 114 indicating that one of the proposed adjustments is approved. Accordingly, the controller 114 may proceed with adjusting one or more of the set-up parameters according to the approved proposal, thereby resulting in the proposed ablation zone being maintained or adjusted (via step 424 of FIG. 4B), thereby allowing the controller 114 to proceed to step 426 (FIG. 4B).

It is noted that, in the example pretreatment images 500, 500′, the controller 114 did not identify any critical structures that are within a desired minimum distance. Accordingly, no adjustment to the image 600 of the proposed ablation zone is required.

At this point, and now referring to FIG. 4B, the controller 114 may then proceed with determining a proposed position of an ablation probe 104 that may generate the proposed ablation zone, as at step 426. Based on determining the orientation and size of the proposed ablation zone, the controller 114 may proceed with determining a position of an ablation probe 104 that may generate the proposed ablation zone.

Briefly referring to FIG. 7, based on determining the position of the ablation probe, the controller 114 may overlay, on the pretreatment image 500, an image 700 representative of the position of the ablation probe that may generate the proposed ablation zone.

The controller 114 may then proceed with determining if the proposed position of the ablation probe is within a threshold distance of, or interferes with, a critical structure and/or bone, as at step 428. Similar to step 418 (FIG. 4A), the controller 114 may determine distances between the proposed position of the ablation probe and the identified critical structures. The controller 114 may retrieve, from the memory 117, the desired minimum distance from critical structures, provided to the controller 114 at step 406 (FIG. 4A), and compare the desired minimum distance to the measured distances.

In addition, the controller 114 may determine if any bones are present within the pretreatment images. The controller 114 may identify these bones using the imaging device 119 or with image recognition software stored in the memory 117. Based on determining the presence of critical structures and/or bones in the patient, the controller 114 may proceed with determining if the proposed position of the ablation probe interferes (e.g. passes through) one or more of the identified critical structures and/or bones.

Based on determining that no critical structures are within the desired minimum distance of the proposed position of the ablation probe and the proposed position of the ablation probe does not interfere with (traverse) any critical structures and/or bones, the controller 114 may proceed to step 436, discussed in more detail below. Based on at least one of the measured distances to a critical structure being at or less than the desired minimum distance and/or the proposed position of the ablation probe interfering with a critical structure and/or bone, the controller 114 may proceed with determining if the proposed ablation zone can be adjusted (e.g. re-oriented and/or re-sized with a different combination of parameters (type of probe, power P, or time T)), as at step 430. Similar to step 420, at step 430, the controller 114 may determine if the proposed ablation zone can be adjusted such that the proposed ablation zone still encompasses, at least substantially encompasses, the desired ablation target and has a boundary that is a distance greater than the desired minimum distance away from the identified critical structures. In addition, the controller 114 may determine if the adjustment to the proposed ablation zone results in a proposed position of an ablation probe that is a distance greater than the desired minimum distance from critical structures and/or bones and that does not interfere with any critical structures and/or bones.

Based on the controller 114 determining that the proposed ablation zone can be adjusted at step 430, the controller 114 may proceed with adjusting the proposed ablation zone as minimally as possible, as at step 434. For instance, referring to FIG. 7, the controller 114 may determine that the proposed position of the ablation probe interferes with (extends through or traverses) a bone 702. Accordingly, the controller 114 may remove the overlays of the images 600, 700, determine an adjustment to the proposed ablation zone and, overlay on the pretreatment image 500, an image 800 representative of an adjusted proposed ablation zone, as shown in FIG. 8.

As discussed above, it should be noted that adjusting the ablation zone may result in a larger proposed ablation zone that may cause additional collateral damage to healthy, non-targeted, tissue. This adjustment, however, may preserve critical structures and/or bones while still encompassing the desired ablation target. Accordingly, the adjustment to the proposed ablation zone may be preferable despite the additional collateral damage to healthy, non-targeted tissue that may be expected.

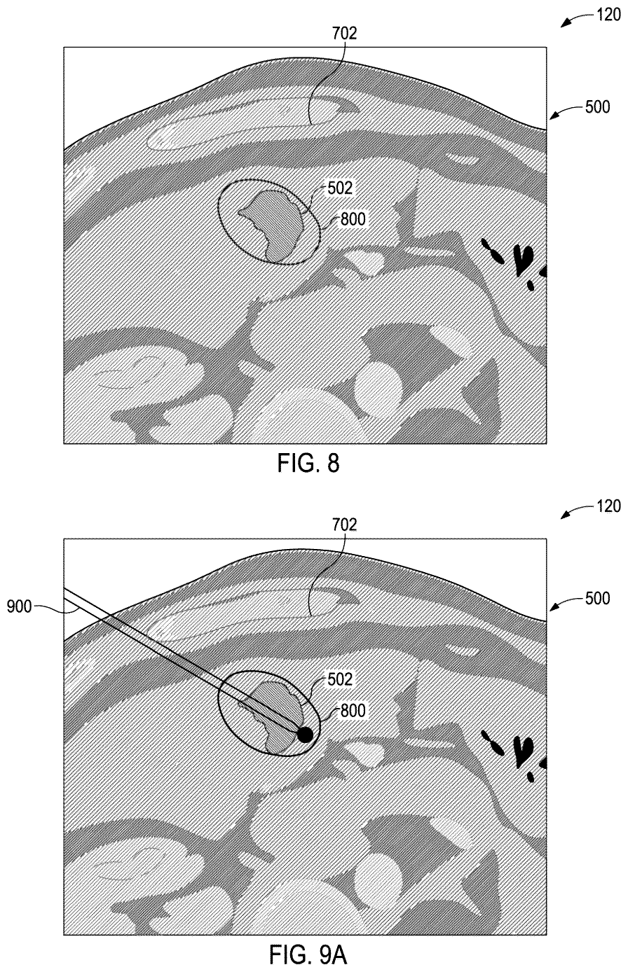

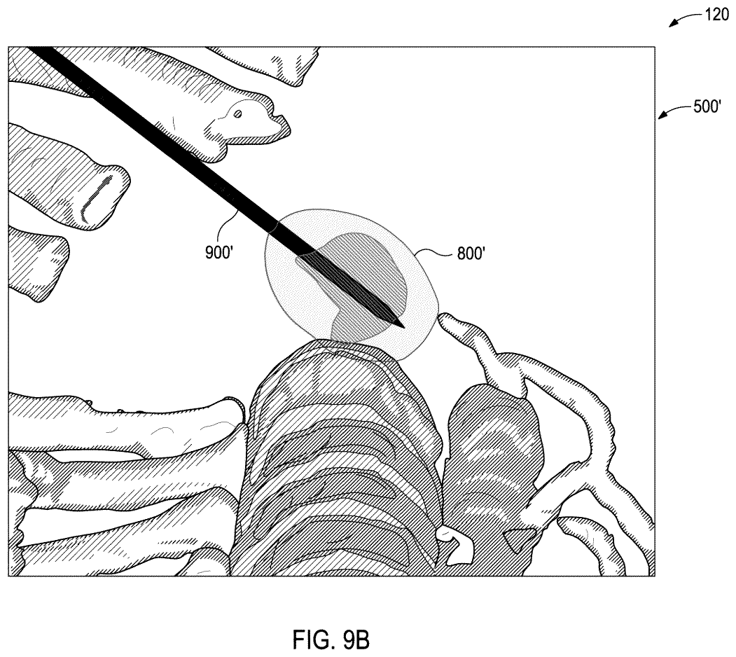

Based on determining an adjustment to the proposed ablation zone, the controller 114 may then proceed with determining a proposed position of an ablation probe that may generate the adjusted proposed ablation zone. With reference to FIG. 9A, based on determining a proposed position of the ablation probe, the controller 114 may overlay on the pretreatment image 500 an image 900 indicative of a newly proposed position of the ablation probe. Notably, the adjusted position of the ablation probe avoids the identified bone 702.

If the controller 114 determines that the proposed ablation zone cannot be adjusted, the controller 114 may proceed with providing an alert, such as a visual alert via the GUI 120, as at step 432. The alert may indicate to a user that an ablation zone cannot be proposed with the provided inputs.

The controller 114 may further determine an adjustment to one or more of the provided set-up parameters (e.g. the margin or the minimum distance) that would allow the controller 114 to continue with the algorithm 400. For instance, the controller 114 may propose, on the GUI 120, a first adjustment (decrease) to one or more of the set-up parameters that would allow the controller 114 to proceed directly to step 436, as shown in FIG. 4B. The controller 114 may also propose a second adjustment (decrease) of one or more set-up parameters different (less) than the first adjustment, thereby allowing the controller 114 to adjust the proposed ablation zone at step 434.

A user may provide an input to the controller 114, via the GUI 120, in response to the proposals provided at step 432. For instance, a user may provide an input to the controller 114 indicating that the proposed adjustments are disapproved. Accordingly, the controller 114 may proceed back to step 410 (FIG. 4A) and indicate to the user, via the GUI 120, that a new desired ablation zone must be drawn to satisfy the desired set-up parameters. Alternatively, the user may provide an input to the controller 114 indicating that one of the proposed adjustments is approved. Accordingly, the controller 114 may proceed with adjusting one or more of the set-up parameters according to the approved proposal, thereby resulting in the proposed ablation zone being maintained or adjusted (via step 434), thereby allowing the controller 114 to proceed to step 436.

The controller 114 may then proceed with providing a message to the user, such as on the GUI 120, indicating that a proposed ablation zone and proposed position of an ablation probe have been prepared and are ready for review and approval, as at step 436. In some embodiments, the controller 114 may display, on the GUI 120, various statistics associated with the proposed ablation zone, as at step 438. The statistics may include the volume of the desired ablation target (i.e. the desired region to ablate plus the desired margin), the volume of the proposed ablation zone, the minimum distance between the perimeter (boundary) of the proposed ablation zone and one or more critical structures and/or bones, the length of the probe insertion (e.g. the distance from the skin to the tip of the stylet 218 (FIG. 2)), the anatomical reference point of entry for the proposed position of the ablation probe (e.g. the bellybutton, between the second and third rib, etc.), or combinations thereof.

Prior to approving the proposed ablation zone and the proposed position of the ablation probe, the user may desire to view an alternative pretreatment image to visualize the proposed ablation zone and proposed position of the ablation probe. As discussed elsewhere herein, a user may provide an input to the controller 114 to transition the GUI 120 between a first state, in which the controller 114 displays, on the GUI 120, the first pre-treatment image (e.g. the 2D image 500 with the overlaid 2D images 502, 800, 900 (FIG. 9A)), and a second state, in which the controller 114 displays, on the GUI 120, a second pre-treatment image (e.g. the 3D image 500′ with corresponding overlaid 3D images 502′, 800′, 900′). Accordingly, a user may transition the GUI 120 between the first and second states to determine if the proposed ablation zone and the proposed position of the ablation probe are satisfactory.

Should a user find the proposed ablation zone and/or the proposed position of the ablation probe unsatisfactory, the user may provide an input to the controller 114, such as via the GUI 120, disapproving the proposed ablation zone and proposed position of the ablation probe. The user may also provide an input to the controller 114, such as via the GUI 120, to manually adjust the proposed position of the ablation probe, as at step 440. The proposed position of the ablation probe may be adjusted by manipulating the proposed position of the ablation probe to an adjusted position and/or orientation, such as via a mouse. Based on manually adjusting the proposed position of the ablation probe to a user adjusted position of the ablation probe, the controller 114 may then proceed back to step 436, requesting approval or disapproval of the updated proposed ablation zone and position of the ablation probe. Based on the user adjustments, the controller 114 may also update the statistics (step 438) as necessary.

Alternatively, at step 436, the user may provide an input to the controller 114 disapproving the proposed ablation zone and proposed position of the ablation probe. Accordingly, the controller 114 may proceed back to step 410 to allow the user to redraw another desired region to ablate.

Based on the controller 114 providing the message at step 436, the user may provide an input to the controller 114 in which the user approves the proposed ablation zone and proposed position of the ablation probe. Accordingly, the algorithm 400 may be finalized and the user may proceed with placing the ablation probe according to the proposals provided by the algorithm 400, as at step 442.

While the foregoing algorithm was provided with respect to placing a single ablation probe, the algorithm 400 may also prepare proposals for a plurality of ablation probes for a single ablation treatment. For instance, the desired region to ablate may be more feasibly ablated by more than one ablation probe. Accordingly, the algorithm 400 may propose using more than one ablation probe and may propose ablation zones and positions of the more than one ablation probe to ablate the desired region to ablate.

Accordingly, the foregoing algorithm 400 may automatically, without user intervention apart from adjustments to user provided parameters when needed, propose ablation zones and proposed positions of ablation probes, thereby greatly simplifying and reducing the time associated with the planning and placement step for an ablation procedure, which may thereby lower the barrier of entry for interventional radiologists and other physicians. The ablation zones proposed by the algorithm minimize collateral tissue damage while still achieving desired margins, such as those set by the user at the outset of the ablation treatment.

Embodiments disclosed herein include:
A. A system comprising an ablation probe that includes an antenna and a controller operable to receive an input indicative of a desired region to ablate with the ablation probe and automatically determine a proposed position of the ablation probe within a patient based on the input.
B. A non-transitory computer readable medium storing instructions that, when executed by a processor, cause the processor to display, on a display, a pretreatment image of a patient, receive an input indicative of a desired region to ablate with an ablation probe, determine a proposed position of the ablation probe within the patient based on the input, and overlay, on the pretreatment image, the proposed position of the ablation probe.
C. A system comprising an ablation probe that includes an antenna and a controller operable to receive an input indicative of a desired region to ablate with the ablation probe, determine a first proposed position of the ablation probe within a patient based on the input, determine an interference with the first proposed position of the ablation probe, and adjust the first proposed position of the ablation probe to a second proposed position of the ablation probe and thereby avoiding the interference.

Each of embodiments A, B, and C may have one or more of the following additional elements in any combination: Element 1: further comprising a display in operable communication with the controller, wherein the controller is further operable to display, on the display, a pretreatment image of the patient and overlay, on the pretreatment image, an image representative of the proposed position of the ablation probe. Element 2: wherein receiving the input comprises receiving a digital drawing of the desired region to ablate on the pretreatment image. Element 3: wherein the input is a first input and the controller is operable to receive a second input indicative of a desired margin, and wherein the controller is further operable to automatically determine the proposed position of the ablation probe based on the second input. Element 4: wherein the controller is further operable to overlay, on the pretreatment image, an image representative of the desired region to ablate and overlay, on the pretreatment image, an image representative of the desired margin around the desired region. Element 5: wherein the controller is further operable to determine a proposed ablation zone generatable by the ablation probe based on the input, and wherein the proposed position of the ablation probe is determined based on the proposed ablation zone. Element 6: wherein the controller is further operable to overlay, on the pretreatment image, the proposed ablation zone. Element 7: wherein the controller comprises a memory, and wherein the controller is further operable to determine the proposed ablation zone by selecting between a plurality of ablation zones stored in the memory. Element 8: wherein the input is a first input and the controller is further operable to receive a second input indicative of a desired minimum distance from a critical structure. Element 9: wherein the proposed position of the ablation probe is a first proposed position of the ablation probe and the controller is further operable to detect the presence of the critical structure within the patient, determine a distance between the critical structure and the proposed ablation zone generatable by the ablation probe in the first proposed position, and adjust the first proposed position of the ablation probe to a second proposed position based on the determined distance being less than the desired minimum distance. Element 10: further storing instructions that, when executed by a processor, cause the processor to receive a second input indicative of a desired margin, wherein the proposed position of the ablation probe is further based on the second input. Element 11: further storing instructions that, when executed by a processor, cause the processor to overlay, on the pretreatment image, an image representative of the desired region to ablate and overlay, on the pretreatment image, an image representative of the desired margin around the desired region. Element 12: further storing a plurality of different sized ablation zones generatable by the ablation probe, wherein to determine the proposed position of the ablation probe within the patient, the processor selects between the plurality of different sized ablation probes. Element 13: further storing instructions that, when executed by a processor, cause the processor to receive a second input indicative of a desired minimum distance from critical structures, wherein the proposed position of the ablation probe is further based on the second input. Element 14: further storing instructions that, when executed by a processor, cause the processor to detect the presence of a critical structure within the patient and adjust the proposed position of the ablation probe to a second proposed position of the ablation probe based on the detected presence of the critical structure and the second input. Element 15: wherein a first amount of tissue is anticipated to be damaged in the first proposed position, and wherein a second amount of tissue greater than the first amount of tissue is anticipated to be damaged in the second proposed position. Element 16: wherein the interference comprises a bone in a path of the first proposed position of the ablation probe. Element 17: wherein the interference comprises a critical structure that is within a threshold distance of a proposed ablation zone generatable by the ablation probe in the first proposed position.

By way of non-limiting example, exemplary combinations applicable to A, B, and C include: Element 1 with Element 2; Element 1 with Element 3; Element 1 with Elements 3 and 4; Element 1 with Element 5; Element 1 with Elements 5 and 6; Element 1 with Elements 5 and 7; Element 1 with Elements 5 and 8; Element 1 with Elements 5, 8, and 9; Element 1 with one or more of Elements 2-9; Element 10 with Element 11; Element 13 with Element 14; Element 10 with one or more of Elements 11-14; Element 12 with one or more of Elements 10, 11, 13, and 14; Element 13 with one or more of Elements 10-12 and 14; Element 14 with one or more of Elements 10-13; Element 15 with one of both of Elements 16 and 17; Element 16 with one or both of Elements 15 and 17; Element 17 with one or both of Elements 15 and 16.

Therefore, the disclosed systems and methods are well adapted to attain the ends and advantages mentioned as well as those that are inherent therein. The particular embodiments disclosed above are illustrative only, as the teachings of the present disclosure may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope of the present disclosure. The systems and methods illustratively disclosed herein may suitably be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the elements that it introduces. If there is any conflict in the usages of a word or term in this specification and one or more patent or other documents that may be incorporated herein by reference, the definitions that are consistent with this specification should be adopted.

As used herein, the phrase “at least one of” preceding a series of items, with the terms “and” or “or” to separate any of the items, modifies the list as a whole, rather than each member of the list (i.e., each item). The phrase “at least one of” allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

The use of directional terms such as above, below, upper, lower, upward, downward, left, right, and the like are used in relation to the illustrative embodiments as they are depicted in the figures, the upward direction being toward the top of the corresponding figure and the downward direction being toward the bottom of the corresponding figure.

What is claimed is:

1. A system, comprising:
an ablation probe that includes an antenna; and
a controller operable to:
receive an input indicative of a desired region to ablate with the ablation probe; and
automatically determine a proposed position of the ablation probe within a patient based on the input.

2. The system of claim 1, further comprising a display in operable communication with the controller, wherein the controller is further operable to:
display, on the display, a pretreatment image of the patient; and
overlay, on the pretreatment image, an image representative of the proposed position of the ablation probe.

3. The system of claim 2, wherein receiving the input comprises receiving a digital drawing of the desired region to ablate on the pretreatment image.

4. The system of claim 2, wherein the input is a first input and the controller is operable to receive a second input indicative of a desired margin, and wherein the controller is further operable to automatically determine the proposed position of the ablation probe based on the second input.

5. The system of claim 4, wherein the controller is further operable to:
overlay, on the pretreatment image, an image representative of the desired region to ablate; and
overlay, on the pretreatment image, an image representative of the desired margin around the desired region.

6. The system of claim 2, wherein the controller is further operable to determine a proposed ablation zone generatable by the ablation probe based on the input, and wherein the proposed position of the ablation probe is determined based on the proposed ablation zone.

7. The system of claim 6, wherein the controller is further operable to overlay, on the pretreatment image, the proposed ablation zone.

8. The system of claim 6, wherein the controller comprises a memory, and wherein the controller is further operable to determine the proposed ablation zone by selecting between a plurality of ablation zones stored in the memory.

9. The system of claim 6, wherein the input is a first input and the controller is further operable to receive a second input indicative of a desired minimum distance from a critical structure.

10. The system of claim 9, wherein the proposed position of the ablation probe is a first proposed position of the ablation probe and the controller is further operable to:
detect the presence of the critical structure within the patient;
determine a distance between the critical structure and the proposed ablation zone generatable by the ablation probe in the first proposed position; and
adjust the first proposed position of the ablation probe to a second proposed position based on the determined distance being less than the desired minimum distance.

11. A non-transitory computer readable medium storing instructions that, when executed by a processor, cause the processor to:
display, on a display, a pretreatment image of a patient;
receive an input indicative of a desired region to ablate with an ablation probe;
determine a proposed position of the ablation probe within the patient based on the input; and
overlay, on the pretreatment image, the proposed position of the ablation probe.

12. The non-transitory computer readable medium of claim 11, further storing instructions that, when executed by a processor, cause the processor to receive a second input indicative of a desired margin, wherein the proposed position of the ablation probe is further based on the second input.

13. The non-transitory computer readable medium of claim 12, further storing instructions that, when executed by a processor, cause the processor to:
overlay, on the pretreatment image, an image representative of the desired region to ablate; and
overlay, on the pretreatment image, an image representative of the desired margin around the desired region.

14. The non-transitory computer readable medium of claim 11, further storing a plurality of different sized ablation zones generatable by the ablation probe, wherein to determine the proposed position of the ablation probe within the patient, the processor selects between the plurality of different sized ablation probes.

15. The non-transitory computer readable medium of claim 11, further storing instructions that, when executed by a processor, cause the processor to receive a second input indicative of a desired minimum distance from critical structures, wherein the proposed position of the ablation probe is further based on the second input.

16. The non-transitory computer readable medium of claim 15, further storing instructions that, when executed by a processor, cause the processor to:
detect the presence of a critical structure within the patient; and
adjust the proposed position of the ablation probe to a second proposed position of the ablation probe based on the detected presence of the critical structure and the second input.

17. A system, comprising:
an ablation probe that includes an antenna; and
a controller operable to:
receive an input indicative of a desired region to ablate with the ablation probe;
determine a first proposed position of the ablation probe within a patient based on the input;
determine an interference with the first proposed position of the ablation probe; and
adjust the first proposed position of the ablation probe to a second proposed position of the ablation probe and thereby avoiding the interference.

18. The system of claim 17, wherein a first amount of tissue is anticipated to be damaged in the first proposed position, and wherein a second amount of tissue greater than the first amount of tissue is anticipated to be damaged in the second proposed position.

19. The system of claim 17, wherein the interference comprises a bone in a path of the first proposed position of the ablation probe.

20. The system of claim 17, wherein the interference comprises a critical structure that is within a threshold distance of a proposed ablation zone generatable by the ablation probe in the first proposed position.
